(19) United States
(12) Patent Application Publication
(10) Pub. No.: US 2020/0368129 A1
(43) Pub. Date: Nov. 26, 2020

(54) COMPOSITION FOR DYEING KERATIN FIBERS CONTAINING A COMBINATION OF A P-PHENYLENE DERIVATIVE AND 2,7-NAPHTHALENEDIOL AND USE THEREOF FOR REDUCING A YELLOW TINGE IN A HAIRSTYLE

(71) Applicant: LABORATOIRE BIOSTHETIQUE KOSMETIK GmbH & Co. KG, Pforzheim (DE)

(72) Inventors: Wolfgang BALZER, Pforzheim (DE); Gunther AUGENSTEIN, Pforzheim (DE); Christian ADER, Pforzheim (DE)

(21) Appl. No.: 16/771,205

(22) PCT Filed: Dec. 21, 2017
(86) PCT No.: PCT/EP2017/084021

§ 371 Date: Jun. 9, 2020

(51) Int. Cl. A61K 8/41; A61K 8/34; A61Q 5/10

(52) U.S. Cl. CPC A61K 8/411; A61K 8/22; A61Q 5/10; A61K 8/347

(57) ABSTRACT

The present invention relates to a composition for dyeing keratin fibers, for example human hair, with oxidative hair dyes. The composition contains as oxidative dyes a combination of a p-phenylenediamine derivative as the developer substance and 2,7-naphthalenediol as the coupler substance in a suitable base. An oxidizing agent such as hydrogen peroxide is used as an additional component for developing the dye. The invention relates to a use of the composition for gray coloration, with a long-lasting reduction of a yellow tinge in a hairstyle of gray, previously dark hair.

### FIELD OF THE INVENTION

The present invention relates to agents for oxidatively dyeing keratin fibers, in particular human hair, which agents contain a combination of one or more p-phenylene derivatives as the developer substances and 2,7-naphthalenediol as the coupler substance. In addition to human hair, animal hair, in particular wool or furs, feathers, nails or artificial keratin fibers can also be dyed, the present invention being directed in particular to dyeing human head hair. The present invention in particular also relates to the use of said agents for dyeing human head hair gray, in particular human hair having a low intrinsic pigment concentration, for the long-lasting reduction of the yellow tinge in a hairstyle containing such hair.

### BACKGROUND OF THE INVENTION

In the field of dyeing keratin fibers, in particular hair dyeing, oxidation colorants have gained great importance. The color is produced here by the reaction of specific developer substances with specific coupler substances in the presence of a suitable oxidizing agent. In this case, in particular 2,5-diaminotoluene, 2,5-diaminophenylethyl alcohol, p-aminophenol, 2-methoxymethyl-1,4-p-phenylenediamine, 1,4-diaminobenzene and 4,5-diamino-1-(2-hydroxyethyl)pyrazole are used as the developer substances, while resorcinol, 2-methylresorcinol, 1-naphthol, 3-aminophenol, 2-amino-4-(2′-hydroxyethyl)aminoanisole and 1,3-diamino-4-(2′-hydroxyethoxy)benzene can be named as the coupler substances.

Furthermore, it is also possible to dye the hair using non-oxidative, substantive dyes containing colorants (often referred to as tints). These colorants, referred to in the following as substantives, do not have the permanency of the colors oxidatively introduced into the hair. Another advantage of the present invention is also that such substantives may no longer be required.

In addition to the stability of the hair colors over a specific time period, numerous additional demands are made on colorants used for dyeing human hair. Therefore, the colorants must be safe from a toxicological and dermatological point of view and the hair colors obtained are intended to have good lightfastness, perm fastness, rubbing fastness and stability against other mechanical strains and standard hair treatments, in particular shampooing, and a sufficient degree of resistance to skin secretions, such as perspiration.

A particular problem is the production of natural shades of gray when creating shades, which are increasingly required in the hairstyle in particular for masking a yellow tinge that often occurs in naturally gray hair having a low intrinsic pigment concentration. In order to achieve natural-looking gray shades, it is necessary to combine a plurality of different oxidation colorants or substantives, which collectively requires a shading of blue, yellow or red colors.

Although products available on the market that are intended to solve this problem and use the above-mentioned oxidation colorants or substantives provide uniform, natural shades of gray after the dyeing process, they display serious weaknesses with regard to permanency. In particular, after approximately 14 days in general, an unwanted change in the shade appears as a result of the action of light, in particular UV light, or as a result of regular shampooing, since individual color components are broken down or washed out.

It has now surprisingly been found that, when using an oxidative combination of a p-phenylenediamine derivative and 2,7-naphthalenediol, the desired permanency when achieving natural shades of gray and the color stability are ensured to a particularly large extent.

Although the use of 2,7-naphthalenediol as the coupler substance, but only ever as part of a mixture with a plurality of other couplers, is described in the literature, there is no suggestion of the improvements in the permanency and color stability of shades of gray. For example, EP 1 707 242 mentions the use of 2,7-dihydroxynaphthaline together with at least one additional coupler, but only in relation to the permanency of intensive colors, without mentioning shades of gray or listing these in the examples at all.

### The Present Invention

The present invention therefore relates to an agent for oxidatively dyeing keratin fibers, such as wool, furs, feathers or hair, and in particular human hair, which is characterized in that said agent contains a combination of at least one p-phenylenediamine derivative as the developer and 2,7-naphthalenediol as the coupler. Furthermore, the present invention is directed to the use of this agent for achieving a long-lasting gray color of hair, in particular hair that is already low in pigment, i.e. gray. In this case, the yellow tinge that often occurs in hairstyles of such hair is masked in a particularly long-lasting manner.

The ready-to-use agent for oxidative dyeing is usually firstly prepared just before use, at least with regard to mixing the dye component and oxidizing agent. If necessary, other components can also be admixed, in particular if the user is intended to be able to choose whether or not they shall be used or in which dosage, for example for agents that affect rheology. Of course, this requires additional packaging. Alternatively, such other components can, however, also be contained in the colorant component or in the oxidizing agent, but they would then no longer be optional. It has proven to be practical and therefore standard to make the separately packaged dye component and the oxidizing agent as well as other components available to the consumer or user.

1,4-diamino-benzene (p-phenylenediamine), 1,4-diamino-2-methylbenzene (p-toluenediamine), 1,4-diamino-2-methoxmethylbenzene, 1,4-diamino-2-hydroxyethylbenzene or N,N-bis-(2-hydroxyethyl)-p-phenylenediamine are preferably considered as the developer substances in the colorant component for the dye in this case, whereby the compounds can be used in the form of their free bases or salts.

In this case, in the colorant component according to the invention, the overall amount of developer or coupler substances can be from approximately 0.05 to 2 percent by weight of the colorant component, in particular from approximately 0.2 to 1 percent by weight of the colorant component.

Furthermore, additional conventional cosmetic additives, for example antioxidants such as ascorbic acid, thiogylcolic acid or sodium sulfite, and perfume oils, complexing agents, wetting agents, emulsifiers, thickeners and conditioning agents can be contained in the colorant component of the dye if said dye is intended to be used for dyeing hair.

The colorant component, according to the invention, of the dye can be prepared in the form of a solution, for example, in particular an aqueous or hydroalcoholic solution, a paste, a cream, a powder, a granulate, a gel, an emulsion, a dispersion, an air-generated foam or an aerosol preparation. Its composition represents a mixture of the colorant components with the additives that are standard in such compositions.

Conventional additives in solutions, creams, emulsions, dispersions or gels are, for example, solvents such as water, lower aliphatic alcohols, for example ethanol, propanol or isopropanol, glycerin or glycols such as 1,2-propyleneglycol, additional wetting agents or emulsifiers from the classes of anionic, cationic, amphoteric or nonionic surface-active substances, such as fatty alcohol sulfates, ethoxylated fatty alcohol sulfates, alkyl sulfonates, alkyl benzene sulfonates, alkyltrimethylammonium salts, alkyl betaines, ethoxylated fatty alcohols, ethoxylated nonylphenols, fatty acid alkanolamides and ethoxylated fatty acid esters as well as thickeners such as higher fatty alcohols, starches, cellulose derivatives, petroleum jelly, paraffin oil and fatty acids, as well as conditioning agents such as cationic resins, lanolin derivatives, cholesterol, pantothenic acid and betaine. The components mentioned are used in the amounts that are conventional for such a purpose, for example the wetting agents and emulsifiers are used in concentrations of from approximately 0.5 to 30 percent by weight of the colorant component, the thickeners in an amount of from approximately 0.1 to 30 percent by weight of the colorant component, and the conditioning agent in a concentration of from approximately 0.1 to 5.0 percent by weight of the colorant component.

Suitable substrates for the powder or granulate applications are powdery, microcrystalline substances that turn the colorant into a physical state, which makes it possible to carry out the method of coating the pellets with suitable encapsulation materials. Suitable substrates are in particular gum arabic, dextrose, polyvinylpyrrolidone, oligosaccharides, microcrystalline cellulose derivatives, such as hydroxypropyl methylcellulose, methylcellulose, hydroxyethyl cellulose, hydroxypropyl cellulose, nonoxynol hydroxyethyl cellulose and cetyl-hydroxyethyl cellulose or physically or chemically modified starches or starch derivates, such as starch esters (for example acetylated starches), starch ethers (for example hydroxyalkylated starches), dialdehyde starches, dicarboxyl starches, distarch phosphates, hydroxyalkyl starch phosphates or hydroxyalkyl starches, wherein the alkyl groups preferably contain from 1 to 4, particularly preferably 2 to 3, C atoms. Also suitable are crosslinked starch ethers, such as those with the INCI designation dimethylimidazolidinone Rice or Corn Starch or hydrophobically modified starches (for example those with the INCI designation Aluminum Starch Octenylsuccinate). The starches can have been both thermally and hydrolytically or enzymatically modified, whereby the starting starch can be obtained from the known sources, for example corn, potatoes, sweet potatoes, peas, bananas, oats, wheat, barley, rice, sago, tapioca, arrowroot, amaranth, canna, sorghum, etc. Particularly preferred starch derivatives are non-ionic starch derivatives, in particular nonionic starch derivatives modified with alkylene oxides such as ethylene oxide, propylene oxide or butylene oxide, acetic anhydride or butyl ketene dimer, and in particular propylene oxide. Additional suitable substrates are synthetic calcium silicate, diatomaceous earth, silicon dioxide or other free-flowing non-caking powders.

Suitable encapsulation materials for the powders or granulates are water-soluble or water-dispersible film-forming substances, which are able to deposit uniform films on the powders from solutions or dispersions by means of spray drying so as to speak of a coating. Suitable encapsulation materials are gum arabic, cellulose derivatives (for example methyl celluloses), polyethylene dispersions, polyacrylic acids, polyvinyl alcohols, polycarbonates, polyvinylpyrrolidone, polyester and polyamides or natural film-formers, such as chitosan, shellac, oligosaccharides or Chinese balsamic resin (colophonium).

Dedusting the powdered compositions can also be achieved using a hydrophobic base consisting of oils and waxes, for example an organically liopohilic compound from the group of vegetable and/or animal fats, oils and waxes, paraffin hydrocarbons, higher alcohols and ethers, aliphatic and aromatic esters and silicon oils.

It is likewise possible to form suspensions that may additionally contain at least one inorganic or organic thickener having a liopohilic character, which form an oleogel or lipogel together with the liopohilic compound.

Vegetable oils, such as jojoba oil; Vaseline; liquid paraffins, for example paraffinum perliquidum and paraffinum subliquidum; silicone oils; liquid, long-chain hydrophobic fatty acid esters, for example octyl palmitate, isocetyl palmitate, isopropyl palmitate and octyl stearate; wax-like, long-chain hydrophobic fatty acid esters and/or synthetic wax substitutes, for example natural or synthetic beeswax (for example Lipowachs 6138G® by Lipo Chemicals), C18 to C36 fatty acids (for example Synchrowachs AW1C® by Croda Chemicals Ltd.), C10 to C36 fatty acid triglycerides, such as octanoic acid/dodecanoic acid triglyceride, hydrated coconut fat acid glycerides (for example Softisan 100® by Hüls AG), glyceryl tribehenate (for example Synchrowachs HRC® by Croda Chemicals Ltd.), fatty acid ester mixtures (for example Cutina B® by Henkel KGaA [partnership limited by shares]), and mixtures thereof are suitable in particular as organically lipophilic compounds. In this case, the use of jojoba oil, fatty acid esters, paraffin oil, combinations of fatty acid esters and paraffin oil and combinations of fatty acid esters and/or paraffin oil with Vaseline is particularly preferable.

The liopohilic compounds are used in a concentration of from approximately 0.1 to 80 percent by weight, preferably from 3 to 65 percent by weight, and in particular from 20 to 50 percent by weight, based on the overall amount of the color component.

Alkali carboxylates, earth alkali carboxylates or aluminum carboxylates, for example sodium palmitate, aluminum/magnesium hydroxystearate or magnesium stearate, aluminum monostearate, aluminum distearate and/or aluminum tristearate; copolymerisates of alkenes, such as ethylene/propylene copolymers; crosslinked organic polymers and lipophilic layered silicates, such as benzyl dimethyl stearyl ammonium hectorite (for example Bentone 28 by Rheox), and mixtures of these lipophilic thickeners can be named in particular as lipophilic inorganic or organic thickeners; wherein alkali stearates, earth alkali stearates, aluminum stearates and aluminum/magnesium hydroxystearates, and in particular magnesium stearates and aluminum stearates, are preferred.

Depending on the composition, the dye according to the invention can react in a weakly acidic, neutral or alkaline manner.

According to the present invention, the dyes can optionally also contain at least one source of ammonia or ammonium ions. Known compounds, for example alkanolamides such as monoethanolamine, diethanolamine, triethanolamine, monopropanolamine, dipropanolamine, tripropanolamine, 2-amino-2-methyl-1,3-propanediol, 2-amino-2-methyl-1-proanol, tris(hydroxymethyl)aminomethane or 2-amino-2-hydroxymethyl-1,3-propanediol can be used as additional alkalizing agents. Particularly preferable alkalizing agents are those agents comprising a source of ammonium ions, in which every source of ammonium ions is suitable. Preferred sources of ammonium ions are ammonium chloride, ammonium sulfate, ammonium nitrate, ammonium phosphate, ammonium acetate, ammonium carbonate, ammonium bicarbonate, ammonium carbamate, ammonium hydroxide, percarbonate salts, ammonia and mixtures thereof. In this case, ammonium carbonate, ammonium carbamate, ammonium bicarbonate, ammonia and mixtures thereof are particularly preferable. The agents according to the present invention can contain from approximately 0.1 to approximately 10 percent by weight, preferably from approximately 0.5 to approximately 5 percent by weight, and in particular from approximately 1 to approximately 3 percent by weight of an alkalizing agent, preferably ammonium ions.

Hydrogen peroxide or its addition compounds of urea, melamine, sodium borate or sodium carbonate in the form of a 3-12%, preferably 6%, aqueous solution, but also atmospheric oxygen, are primarily considered as the oxidizing agent for developing the hair dye.

According to the present invention, the ready-made dyes contain from approximately 0.1 to approximately 12 percent by weight, preferably from approximately 0.5 to 10 percent by weight, and in particular from approximately 1 to approximately 4 percent by weight of the oxidizing agent.

For use with oxidizing agents, the above-described dye is mixed with the oxidizing agent immediately before use and this mixture is applied to the hair in an amount that is sufficient for the hair dyeing treatment—in general approximately 60 to 200 grams, depending on the hair fullness. In this case, the weight ratio between the hair dye and the oxidizing agent can be from 5:1 to 1:3, but preferably from 1:1 to 1:2. Greater amounts of oxidizing agent are predominantly used for higher dye concentrations in the hair dye, or if stronger bleaching of the hair is intended at the same time.

The pH of the ready-made dye is preferably equal to from 4 to 11 and is set when the dye is mixed with the oxidizing agent. If necessary, the pH can be adjusted by adding suitable acids or bases.

The mixture is left on the hair for approximately 3 to 60 minutes at 15 to 50° C., preferably for 30 to 45 minutes; the hair is then rinsed out with water and dried. If necessary, after this rinsing process, the hair is washed with a shampoo and rinsed with a weak organic acid, such as citric acid or tartaric acid, if necessary. The hair is then dried.

The dye according to the invention, which contains a combination of a p-phenylenediamine derivative and 2,7-naphthalenediol, meets the set requirements of achieving natural shades of gray, in particular with regard to the permanency or color stability to a particularly large extent.

Another aspect of the present invention relates to the use of the dyes according to the invention for producing long-lasting, natural shades of gray for masking a yellow tinge in a hairstyle. Such yellow tinges usually occur in gray hair that only contains a few intrinsic pigments, in particular when the intrinsic pigment color was originally in the dark blond to black range. Of course, such hair can be dyed any usual shade, but will then contain a high pigment concentration once again, in which a small amount of intrinsic pigment pairs with a large amount of artificial dye pigments. However, the claims for the current use are not directed to this.

The current use is therefore directed to dyeing gray hair having a slight yellow tinge gray, the natural dark hair color of which is in the range of from dark blond to black and in which the proportion of white, i.e. substantially non-pigmented, hair is greater than 10%, preferably greater than 50% and often more than 85%, but does not reach 100%. The gray proportion of the hair can be visually analyzed by a person skilled in the art, the trained hairdresser, or determined in a laboratory by means of image analysis systems. In this case, the number of hairs that are no longer pigmented with respect to all of the hairs is set and stated as a percent. A dark hair color of the natural hair can be visually determined by a person skilled in the art, the trained hairdresser, or defined in a laboratory by means of the European standard EN ISO 11664-4 “Colorimetry—Part 4: CIE 1976 L*a*b* Colour space.” Such hair typically has lab values that lie in the following ranges: L smaller than 30, a between 0 and 5 and b between 0 and 7.

All of the stated percentages contained in the present application are percents by weight, unless stated otherwise.

The following examples are intended to explain the subject manner of the invention in more detail, without restricting it to these examples.

### EXAMPLES

### Examples 1 to 5: Hair Dye Solution

| | | |
|---|---|---|
| x | g | p-phenyldiamine derivative |
| 0.44 | g | 2,7-naphthalenediol |
| 10.0 | g | potassium oleate (8% aqueous solution) |
| 4.25 | g | monoethanolamine (100%) |
| 10.0 | g | ethanol |
| 0.3 | g | ascorbic acid |
| to 100.0 | g | water |

30 g of the above hair dye solution was mixed with 30 g of a 6% aqueous hydrogen peroxide solution immediately before use. The mixture was then applied to bleached hair. After an application time of 30 minutes at 40° C., the hair was rinsed with water, washed with a commercially available shampoo and dried. The dyeing results are summarized in Table 1.

TABLE 1
color results from dye solution

| | p-phenylene | | |
|---|---|---|---|
| Example | derivative | Conc. (x) | Color result |
| 1 | 1,4-diamino-benzene | 0.295 g | Gray (slightly violet) |
| 2 | 1,4-diamino-2-methyl- | 0.600 g | Steel gray |
| | benzene sulfate | | |
| 3 | 1,4-diamino-2- | 0.415 g | Gray (slightly |
| | methoxymethylbenzene | | reddish) |
| 4 | 1,4-diamino-2- | 0.683 g | Gray (slightly |
| | hydroxyethyl-benzene | | reddish) |
| 5 | N,N-Bis-(2- | 0.852 g | Gray (slightly |
| | hydroxyethyl)-p- | | turquoise) |
| | phenylenediamine | | |
| | sulfate | | |

### Examples 6 to 8: Color Stabilities

### Inventive Product (IP)

| | | |
|---|---|---|
| 0.600 | g | p-phenylenediamine derivative |
| 0.44 | g | 2,7-naphthalenediol |
| 15.0 | g | cetyl alcohol |
| 0.3 | g | ascorbic acid |
| 3.5 | g | sodium lauryl alcohol diglycol ether |
| | | sulfate, 28% aqueous solution |
| 3.0 | g | ammonia, 22% aqueous solution |
| 0.3 | g | sodium sulfite, anhydrous |
| to 100.0 | g | water |

30 g of the above hair dye cream was mixed with 30 g of a 6% hydrogen peroxide solution immediately before use. The mixture was then applied to the hair. After an application time of 20 minutes, the hair was rinsed with water, washed with a commercially available shampoo and dried.

For comparison, three market products from the IGORA ABSOLUTES SILVERWHITE range by Henkel, Dusseldorf, Germany are likewise used to color hair according to the instructions for use:

### Market Product 1 (MP1): Gray Lilac

According to the INCI, colorants as the developer are: 1,4-diamino-2-methyl-benzene sulfate and N,N-Bis-(2-hydroxyethyl)-p-phenylenediamine sulfate, and as the coupler are: resorcinol, 4-chlororesorcinol, 2-methylresorcinol, m-aminophenol and 4-amino-2-hydroxytoluene

### Market Product 2 (MP2): Dove Gray

According to the INCI, colorants as the developer are: 1,4-diamino-2-methyl-benzene sulfate and N,N-Bis-(2-hydroxyethyl)-p-phenylenediamine sulfate, and as the coupler are: resorcinol, m-aminophenol and 2,7-naphthalenediol

### Market Product 3 (MP3): Slate Gray

According to the INCI, colorants as the developer are: 1,4-diamino-2-methyl-benzene sulfate and N,N-Bis-(2-hydroxyethyl)-p-phenylenediamine sulfate, and as the coupler are: resorcinol, m-aminophenol and 2,7-naphthalenediol.

### Testing Light Stability

The laboratory measured values were determined from the strands (yak hair) treated using the four products (IP, MP1, MP2, MP3). The strands were then exposed to sunlight for three days. After this time, the lab values were re-measured. The results are shown in Table 2.

### Testing Wash Stability

The laboratory measured values were determined from the strands (bleached, blond natural hairs) treated with the four products (IP, MP1, MP2 MP3). The strands were then added to a 20% shampoo solution for 24 h. The strands were then rinsed with water and dried. The lab values were then re-measured.

The results are shown in Table 3.

### Testing Carrying Stability on the Model

The formula according to the invention was compared with the formula MP3 in a half-head test on a model (medium-blond initial hair color, 80-90% gray proportion). After the use according to the instructions for use, the lab values were measured at 10 points in each case and an average was generated.

After 14 days the values were measured again.

The results are shown in Table 4.

TABLE 2
color stability after the action of light

| | Starting values | | | Values after treatment | | | | | | |
|---|---|---|---|---|---|---|---|---|---|---|
| Product | L | a | b | L | a | b | ΔL | Δa | Δb | ΔE |
| IP | 33.86 | 0.06 | 0.77 | 35.38 | 1.90 | 2.52 | 1.52 | 1.84 | 1.75 | 2.96 |
| MP1 | 39.72 | 1.33 | −4.10 | 44.37 | 0.92 | 0.24 | 4.65 | −0.41 | 4.34 | 6.37 |
| MP2 | 41.60 | −1.60 | −2.66 | 42.57 | −1.08 | 3.09 | 0.97 | 0.52 | 5.75 | 5.85 |
| MP3 | 30.65 | 1.30 | −2.15 | 27.09 | 0.70 | 1.10 | −3.56 | −0.60 | 3.25 | 4.86 |

The result shows that the formula according to the invention (IP) has a substantially lower overall color deviation (SE) and therefore better stability against the action of light.

TABLE 3
color stability after the action of shampoo

| | Starting values | | | Values after treatment | | | | | | |
|---|---|---|---|---|---|---|---|---|---|---|
| Product | L | a | b | L | a | b | ΔL | Δa | Δb | ΔE |
| IP | 23.17 | 4.36 | −2.82 | 23.74 | 4.86 | −3.74 | 0.57 | 0.50 | −0.92 | 1.19 |
| MP1 | 35.64 | 1.25 | −0.66 | 42.42 | 1.16 | 0.23 | 3.78 | −0.09 | 0.89 | 6.84 |
| MP2 | 39.00 | 1.56 | 2.25 | 43.66 | 2.82 | 2.39 | 4.66 | 1.26 | 0.15 | 4.83 |
| MP3 | 2.775 | 2.07 | 1.35 | 36.66 | 2.41 | 3.91 | 4.35 | 0.08 | 0.51 | 4.38 |

The result shows that the formula according to the invention (IP) has a substantially lower overall color deviation (ΔE) and therefore better stability against the action of shampoo.

TABLE 4
color stability on the model

| | Starting values | | | Values after treatment | | | | | | |
|---|---|---|---|---|---|---|---|---|---|---|
| Product | L | a | b | L | a | b | L | a | b | E |
| IP | 46.36 | 3.02 | 6.23 | 46.15 | 3.78 | 9.76 | −0.21 | 0.76 | 3.53 | 3.61 |
| MP3 | 60.57 | 3.00 | 7.34 | 52.63 | 2.89 | 8.34 | −7.64 | −0.10 | 1.00 | 8.00 |

### Hairdressers' Evaluation of the Invention

For the hairdressers' evaluation, 2 models were each treated with the gray color according to the invention and a market product. The conventional half-head treatment was not possible in this case, since the difference in the result was so significant and easily noticeable that suitable models were not available therefor until a comparative color was offered for the market product in a reduced portion of the head (in this case, the above-mentioned MP3). The resultant results are therefore not classical half-head comparisons, but comparisons between a full-head treatment with the inventive dye, leaving a partial surface of the head, and treatment with MP3 in this partial surface.

The hairdressers' evaluation was carried out by 3 experienced hairdressers with over 10 years' experience in each case in dyeing work using test models and the conventional techniques for evaluating hair color results.

### Hair Condition Before Dyeing

Model H: natural tone of the hair 5/0, medium brown, 100% white proportion on the contour, 50% on the top of the head and 30% on the back of the head. Hair length up to 30 cm. Section for the MP3 comparative color on the back of the head. A very strong and clearly noticeable yellow tinge effect of the hair was confirmed by all the hairdressers and was particularly noticeable in the white proportion of the hair.

Model Z: natural tone of the hair 4/0, medium brown, 100% white proportion on the contour, 25% on the top of the head and 20% on the back of the head. Hair length up to 25 cm. Section for the MP3 comparative color on the back of the head. A strong and clearly noticeable yellow tinge effect of the hair was confirmed by all the hairdressers, particularly in the white proportion of the hair, which was less evident as a result of the smaller white proportion compared with Model H.

### Dye Use

For the color as per the invention, the hair dye cream mentioned under IP was prepared and used as stated below, while, for comparison, the hair dye cream prepared from the market product MP3 was used according to the instructions for use.

In order to prepare the ready-made dye, the IP cream was diluted with a 1.9% peroxide solution in a ratio of 1 to 4 and mixed therewith. The resultant dye was applied to the hair using a brush and left for 20 minutes, then rinsed with water, washed with a commercially available shampoo and dried.

### Evaluation and Results:

The evaluation follows the German school grading principle, with 1 as very good, 2 as good, 3 as satisfactory, 4 as sufficient, 5 as poor and 6 as insufficient. The evaluation by the hairdressers relates to the quality of the dyeing result including the perceived reduction in or elimination of a yellow tinge that was clear prior to dyeing.

### Evaluation of the Color According to the Invention for Model H

| | Before | Day of | After 7 | After 14 | After 21 | After 28 |
|---|---|---|---|---|---|---|
| Hairdresser | dyeing | dyeing | days | days | days | days |
| Miss L. | 4.5 | 2.0 | 2.0 | 2.0 | 2.0 | 2.5 |
| Mr B. | 4.5 | 2.5 | 2.5 | 2.5 | 3.0 | 3.0 |
| Mr H. | 4.3 | 2.0 | 2.0 | 2.5 | 2.5 | 2.5 |
| average | 4.5 | 2.1 | 2.1 | 2.3 | 2.5 | 2.7 |

### Evaluation of the Comparative Color for Model H

| | Before | Day of | After 7 | After 14 | After 21 | After 28 |
|---|---|---|---|---|---|---|
| | dyeing | dyeing | days | days | days | days |
| Miss A L. | 4.0 | 3.5 | 3.5 | 3.5 | 4.0 | 4.0 |
| Mr B B. | 4.5 | 3.5 | 3.5 | 4.0 | 4.0 | 4.0 |
| Mr S H. | 4.3 | 3.0 | 3.5 | 4.0 | 4.0 | 4.0 |
| average | 4.3 | 3.3 | 3.5 | 3.8 | 4.0 | 4.0 |

### Noticeability of the Yellow Tinge in Model H

| | 1 | 2 | 3 | | |
|---|---|---|---|---|---|
| | “not | “difficult | “easy | 4 | |
| | notice- | to | to | “very | 5 |
| Agent | able” | notice” | notice” | visible” | “obvious” |
| Color | — | SH, BB, AL | — | — | — |
| according | | | | | |
| to the | | | | | |
| invention | | | | | |
| Comparative | — | — | SH, BB, AL | — | — |
| dyeing | | | | | |
| process | | | | | |

### Evaluation of the Color According to the Invention for Model Z

| | Before | Day of | After 7 | After 14 | After 21 | After 28 |
|---|---|---|---|---|---|---|
| Hairdresser | dyeing | dyeing | days | days | days | days |
| Miss L. | 3.5 | 2.0 | 2.0 | 2.0 | 2.0 | 2.5 |
| Mr B. | 4.0 | 2.5 | 2.5 | 2.5 | 3.0 | 3.0 |
| Mr H. | 4.5 | 1.5 | 2.0 | 2.5 | 2.5 | 2.5 |
| average | 4.0 | 2.0 | 2.2 | 2.3 | 2.5 | 2.8 |

### Evaluation of the Comparative Color for Model Z

| | Before | Day of | After 7 | After 14 | After 21 | After 28 |
|---|---|---|---|---|---|---|
| | dyeing | dyeing | days | days | days | days |
| Miss A L. | 3.5 | 3.5 | 3.5 | 4.0 | 4.0 | 4.5 |
| Mr B B. | 4.0 | 3.0 | 3.0 | 3.5 | 4.0 | 4.0 |
| Mr S H. | 4.5 | 2.5 | 3.0 | 3.5 | 4.0 | 4.0 |
| average | 4.0 | 3.0 | 3.2 | 3.7 | 4.0 | 4.2 |

### Noticeability of the Yellow Tinge in Model Z

| | 1 | 2 | 3 | | |
|---|---|---|---|---|---|
| | “not | “hard | “easy | 4 | |
| | notice- | to | to | “very | 5 |
| Agent | able” | notice” | notice” | visible” | “obvious” |
| Color | — | SH, BB, AL | — | — | — |
| according | | | | | |
| to the | | | | | |
| invention | | | | | |
| Comparative | — | BB, AL | SH | — | — |
| dyeing | | | | | |
| process | | | | | |

It is clearly apparent from these random results that both the quality of the color according to the invention and the permanency thereof has improved considerably. Furthermore, it is apparent that a yellow tinge is almost eliminated and does not reappear as strongly, even after 4 weeks, due to the quality of the dyeing process.

1. An agent for oxidatively dyeing keratin fibers, characterized in that said agent contains a combination of a p-phenylenediamine derivative and 2,7-naphthalenediol as the sole couplers or the physiologically compatible water-soluble salts thereof.

2. The agent as per claim 1, characterized in that the p-phenylenediamine derivative is selected from 1,4-diaminobenzene (p-phenylenediamine), 1,4-diamino-2-methylbenzene (p-toluenediamine), 1,4-diamino-2-methoxymethylbenzene, 1,4-diamino-2-hydroxyethylbenzene or N,N-bis-(2-hydroxyethyl)-p-phenylenediamine, or mixtures thereof.

3. The agent of claim 1, characterized in that said ready-made agent contains the p-phenylenediamine derivative and the 2,7-naphthalenediol in an overall amount of from 0.05 to 2 percent by weight in each case.

4. The agent of claim 1, characterized in that said ready-made agent contains the p-phenylenediamine derivative and the 2,7-naphthalenediol in an overall amount of from 0.2 to 1 percent by weight in each case.

5. The use of the agent of claim 1 for dyeing human hair gray, the natural color of which hair is dark blond to black and the proportion of white hair of which is more than 10%.

6. The use as per claim 5, wherein the hair has never before been dyed prior to being dyed gray and therefore only contains intrinsic pigments of the user.

7. The use as per claim 6, wherein the hair has a yellow tinge before being dyed gray, which is no longer noticeable after being dyed.
